(19) United States
(12) Patent Application Publication
(10) Pub. No.: US 2026/0294777 A1
(43) Pub. Date: Oct. 1, 2026

(54) COMPOSITIONS CONTAINING RETINOIC ESTERS HAVING EXTENDED STORAGE STABILITY AND COSMETIC COMPOSITIONS CONTAINING THE SAME

(71) Applicant: Grant Industries, Inc., Elmwood Park, NJ (US)

(72) Inventors: Kaylee Gaspar, Elmwood Park, NJ (US); Sandip K. Nandy, Ridgewood, NJ (US); Ronald V. Lerum, Leonia, NJ (US)

(73) Assignee: Grant Industries, Inc., Elmwood Park, NJ (US)

(21) Appl. No.: 19/631,374

(22) Filed: Mar. 27, 2026

Related U.S. Application Data
(60) Provisional application No. 63/779,541, filed on Mar. 28, 2025.

(51) Int. Cl. A61K 8/67; A61K 8/73; A61K 9/00; A61K 31/07; A61K 47/14; A61K 47/26; A61K 47/40; A61Q 19/08

(52) U.S. Cl. CPC A61K 8/671; A61K 8/738; A61K 9/0014; A61K 31/07; A61K 47/14; A61K 47/26; A61K 47/40; A61Q 19/08; A61K 2800/33; A61K 2800/522

(57) ABSTRACT

Retinoid-containing compositions that include hydroxypinacolone retinoate (HPR) complexed with a cyclodextrin demonstrating enhanced long-term stability are described as well as methods of making the same and cosmetic compositions including the same. The compositions may further include an antioxidant and a bioactive enhancer such as a bakuchiol ester.

### CROSS-REFERENCE TO RELATED APPLICATIONS

This application claims the benefit of priority from U.S. Provisional Patent Application Ser. No. U.S. 63/779,541 filed Mar. 28, 2025, the contents of which are incorporated herein by reference.

### FIELD OF THE INVENTION

The invention relates to topical compositions comprising a combination of a retinoic ester, such as hydroxypinacolone retinoate (HPR) and a cyclodextrin, and optionally an antioxidant. These compositions are particularly suitable for pharmaceutical or cosmetic formulations, with a focus on cosmetic applications. It has been found that the combination of HPR and cyclodextrins is effective in preventing or treating age- and stress-related signs of skin aging. Furthermore, the compositions provide enhanced stability, non-cytotoxicity and efficacy of the retinoid when applied to the skin.

### BACKGROUND OF THE INVENTION

Skin aging, both intrinsic and extrinsic, is characterized by the appearance of fine lines, wrinkles, loss of elasticity, and other visible signs of aging. Factors such as environmental stressors, ultraviolet (UV) radiation, and intrinsic biological processes contribute to these visible signs. The effects of sunlight are known to be detrimental to the skin by the production of radical oxygen species (ROS). ROS are a byproduct of several metabolic pathways and enzymatic reactions, and they are classified as radicals, including superoxide anion; hydroxyl radical as well as nonradical species such as hydrogen peroxide and singlet oxygen. ROS has been identified to cause a cascade of negative effects on cellular functions by interfering with DNA, proteins, lipids and destroying important ligands (i.e. Retinoids). These high energy species wreak havoc on DNA by breaking the DNA strands and mutating the bases. ROS leads to breakage of proteins and effects protein conformations leading to dysfunctional enzymes. On lipids, ROS can break lipid chains and destroy membrane fluidity and rigidity. Although ROS may cause a series of unwanted effects, ROS has also played an important role in signaling stimuli responsive genes in the cell. Mediating the balance of ROS production and elimination is necessary for normal cellular behavior. To maintain the balance of cellular homeostasis, there are two main pathways-1) an enzymatic approach and 2) non-enzymatic antioxidants. Enzymatic quencher (endogenous antioxidant) involves the use of superoxide dismutase (SOD), glutathione-S-transferase (GST), catalase (CAT), etc. Enzymatic antioxidants are localized and limited to compartments within the cellular machinery. Examples of non-enzymatic (exogenous antioxidant) small molecules are flavonoids, glutathione, proline, ascorbic acid, etc. These non-enzymatic antioxidants may be present intracellularly and extracellularly, thereby providing a potentially vital role in skin rejuvenation and ROS elimination.

Among the exogenous antioxidants, flavonoids are heavily used and widely distributed in plants. Flavonoids belong to the polyphenol family and can be visualized as two benzene rings which are joined together with a short three carbon chain. Flavonoids exist as an aglycone or glycoside form and as their methylated derivatives. The C ring of the general backbone of the flavonoid allows for the classification into major subgroups: flavanones, flavones, flavonols, flavanols, isoflavones, chalcones, and anthocyanins. These subgroups can be substituted with sugars and hydroxyl groups to increase the water solubility while other substituents, such as alkyl and methyl groups increase the lipophilicity of the flavonoids. There are over 6000 naturally occurring flavonoids that have been identified. The health benefits of flavonoids have been popularized to potentially have anti-cancer, anti-inflammatory, antioxidant and anti-viral properties. In addition, there have been reports of flavonoids with the ability to inhibit Cytochromes (CYP) P450 activity. CYP P450 are a superfamily of enzymes in mammals that oxidize steroids, fatty acids, and xenobiotics to allow the metabolic breakdown, inactivation and clearance of various compounds. Therefore, flavonoids have the potential to inhibit the CYP P450 enzymes involved in the metabolic breakdown of retinoids.

Flavonoids, recognized for their antioxidant and anti-inflammatory properties, offer significant potential for skincare applications. However, their use is restricted by limitations such as poor water solubility, low bioavailability, and rapid metabolism (fast-pass), which reduce their efficacy when applied topically or ingested. The present disclosure outlines a novel composition that enables enhanced delivery of flavonoids, overcoming these limitations and improving their effectiveness. The general core structure of flavonoids and numbering scheme are shown below:

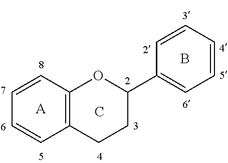

Retinoids and their derivatives are widely used in the cosmetics industry as anti-aging compounds. However, problematically, after longer exposure with compositions of prior art, skin often reacts with inflammation, allergy, redness, peeling, dryness, itch or UV protection weakness.

Retinoids, such as hydroxypinacolone retinoate (HPR), have been widely studied for their ability to address these issues without irritation or sensitization. However, there remains a need for effective topical formulations that combine HPR with cyclodextrins and possibly bioactive agents to enhance their stability as well as effectiveness in treating and preventing signs of skin aging. In the present invention, the inventors tried to find a solution that copies all positive effects of retinoid without the negative side effects that arise due to longer exposure of the actives.

Retinoids (Vitamin A and derivatives) are fat-soluble, lipophilic molecules that can come from natural sources or synthetically prepared. Retinoids are actives that bind specifically to the retinoid acid receptor elements (RARE) in the nucleus of cells. Under typical biological conditions the three retinoids (retinyl palmitate, retinol, and retinal) could bind to the retinoid receptor but conversion to retinoic acid by one or more conversions is preferred although time consuming and may have poor conversion. Efficacy is inevitably decreased during this conversion as the Vitamin A derivatives are competitively metabolized or oxidized in the cell. HPR is already in the correct oxidation state to bind directly to the RAR-alpha and initiate RARE activity. The three receptors of interest are the retinoic acid receptor (RAR)-alpha, -beta, and -gamma. Retinoid derivatives typically consist of retinoic acid, retinaldehyde, retinol, retinoic acid esters (i.e. hydroxypinacolone retinoate, retinyl retinoate, etc.), and esters of retinols (i.e. retinyl palmitate, retinyl propionate, retinyl acetate, etc.). Retinoids have been studied extensively for several decades and have been found to be important as key development regulators for physiological functions such as eye health, immune health, reproductive health, skin health, and healthy growth. As a major key regulator in skin health, retinoids have been extensively used in the treatment of skin disorders ranging from acne to skin aging. Retinoids are formulated into topical cosmetic formulations to repair and return the health of aging skin by the activation of nuclear receptors. More specifically, retinoids have been identified to participate in a number of skin cellular processes, i.e., cell differentiation, cell proliferation, skin rejuvenation, skin turnover, reduction of age spots, skin elasticity, and skin integrity.

Nuclear receptors are a subtype of intracellular receptors located inside the nucleus of the cell. Nuclear receptors function as transcription factors and alter gene transcription, whereby ligands bind to receptors, bind to the DNA, and influence the expression of specific genes. Some examples of nuclear receptors include glucocorticoid receptor, retinoid receptors, Vitamin D receptor, liver X receptor, hormonal receptors, and peroxisome proliferator-activated receptors.

The Retinoic Acid Receptor (RAR) forms heterodimers with the Retinoid X Receptors (RXR) that are involved in DNA transcriptional skin rejuvenation. The activation of this receptor elucidates hundreds of regulatory genes and pathways. The RAR does not form dimers with itself. The activation of the RXR alone is not enough to promote the transcription process. The binding of Retinoic acid and analogs to the RAR is able to mediate the response of the transcription factors.

Another nuclear receptor of interest in skin care pertains to peroxisome proliferator-activated receptors (PPAR). PPAR is comprised of three different isoforms, namely PPAR-alpha, PPAR-gamma and PPAR-beta/gamma with PPAR-beta/gamma predominantly occurring in human keratinocytes. Binding with specific ligands allows the regulation of gene expression, cellular growth and differentiation, apoptosis and inflammatory responses. These responses effectively modify skin function including proliferation, epidermal barrier formation and skin permeability, wound healing and barrier repair, melanocyte proliferation, and sebum production. While the ligands of PPAR are still not fully elucidated, PPARs are activated by a variety of ligands that are typically from the metabolism of fatty acids (FAs), such as unsaturated fatty acid esters (oleates, linoleates, linolenates, etc.). These fatty acid esters may be esterified with hydrophilic groups (i.e. glyceryl) or hydrophobic groups (i.e. alkyl functionality).

Furthermore, stability in a delivery vehicle that promotes uniform dosing of an active by penetration into the skin is highly beneficial for treating sensitive skin, skin ageing and skin disorders. A time-release and uniform delivery can be achieved by enhancing lipophilic properties of the active to enhance formulation compatibility and enhancing molecular properties to allow skin penetration followed by hydrolysis of the active. Bakuchiyl Salicylate in Vitamin F glyceryl ester solvents have been shown to demonstrate this behavior.

### SUMMARY OF THE INVENTION

In one aspect of the invention, there are provided retinoid-containing compositions which include a retinoid complexed within a cyclodextrin. The retinoid is preferably hydroxypinacolone retinoate (HPR) and is encapsulated by (i.e., complexed within) the cyclodextrin. The retinoid-containing compositions exhibit surprisingly extended storage stability (i.e., improved-shelf life) while at the same time maintaining low levels of cytotoxicity.

Cyclodextrin complexes are supramolecular structures formed by encapsulating a hydrophobic guest molecule within the hydrophobic cavity of a toroidal-shaped cyclodextrin molecule. Cyclodextrin (CD) encapsulation is a supramolecular technique using cyclic oligosaccharides to create host-guest inclusion complexes. These bucket-shaped molecules, with hydrophobic cavities and hydrophilic exteriors, trap small organic compounds. The invention also provides novel topical compositions comprising a combination of a retinoid (hydroxypinacolone retinoate (HPR)) complexed within cyclodextrin, and an antioxidant. The compositions can optionally include at least one further bioactive component, and/or an excipient. These compositions are formulated as pharmaceutical or cosmetic products with specific focus on their application in cosmetic treatments aimed at reducing signs of skin aging. The combination of HPR complexed within cyclodextrin with possible bioactive ingredients has been shown to provide enhanced benefits in preventing or treating age-related skin changes, including the reduction of fine lines, wrinkles, and skin texture improvement.

Retinoids are lipophilic compounds and typically precipitate or crystallize out of formulations with high water content (>60%). As a result of the present invention, the use of nitrogen for storage and application which was previously highly recommended to prevent oxidative degradation is no longer needed.

In view of the above, the invention provides several embodiments illustrating the advantages of the invention. In one embodiment, the invention provides a retinoid-containing composition with extended storage stability comprising: the HPR retinoid complexed within a cyclodextrin; and optionally, an antioxidant, wherein the retinoid retains at least 92.3% activity as measured by a Retinoic Acid Receptor element assay based on luciferase activity of the retinoid after 4 weeks at 50 μM HPR under accelerated storage conditions of 50° C., exposed to air and devoid of light. In a particular embodiment, the cyclodextrin is gamma (γ) cyclodextrin. In an embodiment, the antioxidant can be a flavonoid. In another embodiment, the flavonoid is selected from among quercetin, rutin, glutathione, tocopherols (Vitamin E), ascorbic acid (Vitamin C), butylated hydroxyanisole (BHA), butylated hydroxytoluene (BHT), uric acid, and ubiquinol. In a particular embodiment, the flavonoid is alpha-glucosyl rutin (AGR). In accordance with the invention, the HPR-containing composition with improved shelf-life is a water dispersible composition without surfactants. In another embodiment, the retinoic acid compositions maintain residual activity content without the use of a container with inert atmosphere storage.

As illustrated below, the concentration of the components of the retinoid-containing composition with improved shelf-life is variable. For example, in one embodiment, the amount of retinoic acid ester complexed with the cyclodextrin is from about 0.01% to about 25% by weight of the composition. In another embodiment, the retinoic acid ester complexed with the cyclodextrin is in an amount from about 1% to about 13% by weight of the composition. In another embodiment, the cyclodextrin is in an amount from about 80% to about 98% by weight of the composition. In further embodiment, the amount of cyclodextrin is from about 85 to about 95% by weight of the composition. In another embodiment, when included, the antioxidant is present in an amount from about 1% to about 10% by weight of the composition. In another embodiment, the amount of antioxidant is from about 3% to about 8% by weight of the composition.

In one particular embodiment, the retinoid-containing composition with improved shelf-life includes hydroxypinacolone retinoate (HPR), and the cyclodextrin is gamma (v) cyclodextrin. The compositions further include the antioxidant flavonoid alpha-glucosyl rutin (AGR). In a particular embodiment, the HPR is present in an amount of 0.01-25% by weight of the composition, preferably 1-10%, the γcyclodextrin is present in an amount of 75-99.99% by weight of the composition, preferably 85-99% and the AGR is present in an amount of 1-10% by weight, preferably 3-8%.

In another embodiment, the invention provides a topical cosmetic or pharmaceutical composition including the retinoid-containing composition with improved shelf-life described above. In one embodiment, the retinoid-containing composition is in an amount from about 0.001% to about 1% by weight of the topical cosmetic or pharmaceutical composition. In another embodiment, the amount of the retinoid-containing composition is from about 0.01% to about 0.5% by weight of the topical cosmetic or pharmaceutical composition.

In a particular embodiment, the topical cosmetic or pharmaceutical composition further includes a bioactive enhancer. In one embodiment, the bioactive enhancer is a bakuchiol ester. In a further embodiment, the bakuchiol ester is bakuchiol salicylate. In another embodiment, the bioactive enhancer is in an amount from about 0.005% to about 0.3% by weight of the composition.

These and other unique aspects of the invention will become more readily apparent from the detailed description set forth below.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is photograph of three (3) test tubes illustrating dispersibility in water for HPR with excipients. From left to right, the photograph shows HPR in DMI, Example 2-HPR complexed in gamma-cyclodextrin, and Comparative Example 16-HPR blended with gamma-cyclodextrin, taken at 25° C. for T=0;

FIG. 2 is a photograph of two (2) of the test tubes shown in FIG. 1 illustrating dispersibility in water for HPR with excipients after a time-lapse. From left to right, the photograph shows the test tubes for Example 2-HPR complexed in gamma-cyclodextrin, and HPR in DMI taken at 85° C. for T=15 minutes; and

FIG. 3 is a photograph of three (3) test tubes shown in FIG. 1 illustrating dispersibility in water for HPR with excipients after a further time-lapse. From left to right, the photograph shows the test tubes for Example 2-HPR complexed with gamma-cyclodextrin, HPR in DMI, and Comparative Example 16-HPR blended with gamma-cyclodextrin, taken at 85° C. for T=2.5 hours.

### DETAILED DESCRIPTION OF THE INVENTION

Skin acts as a barrier between the body and the external environment, playing a vital role in protection and appearance. As the body ages, the skin undergoes visible changes, which result from a combination of intrinsic and extrinsic factors. These age-related alterations are a driving force behind the global demand for cosmetic products and pharmaceutical interventions designed to either prevent or reverse the effects of skin aging.

Intrinsic Aging: Intrinsic aging, often referred to as chronological aging, is a natural process influenced by genetic and metabolic factors. It is characterized by physiological changes such as thinner, drier skin, fine wrinkles, and dermal atrophy. One of the most notable changes occurs within the basal cell layer, where there is a reduction in cell proliferation, leading to thinning of the epidermis and diminished contact between the dermis and epidermis. This reduced contact surface weakens the skin's ability to nourish itself, exacerbating the appearance of aging. Furthermore, the dermis undergoes several structural changes, including a decline in mast cells, fibroblasts, and collagen fibers, which are essential for skin strength and elasticity. As collagen production decreases, the skin's ability to retain water diminishes, resulting in a loss of structural integrity and an overall decrease in skin hydration.

Extrinsic Aging: Extrinsic aging is primarily caused by UV radiation, which accelerates skin aging. UV exposure thickens the epidermis, especially the stratum corneum, and impairs the differentiation process of epidermal cells. Moreover, UV radiation damages the dermal-epidermal junction, leading to decreased production of type VII collagen, which weakens the skin's structural connections and contributes to wrinkles. The increased degradation of collagen due to elevated matrix metalloproteinases (MMPs) further deteriorates skin structure. Solar elastosis, a hallmark of photoaged skin, results from the accumulation of abnormal elastic fibers in the dermis, caused by UV-induced degradation. This leads to the disruption of skin elasticity.

A youthful and attractive appearance can positively influence social interactions. However, the aging process begins at birth, and the skin, being the body's largest organ, is no exception. As individuals age, the skin becomes one of the most visibly affected organs, displaying clear signs of aging. Consequently, many individuals, particularly women, allocate a significant portion of their daily expenditures towards cosmetics and pharmaceuticals aimed at preventing or reversing these aging effects. This substantial demand has spurred ongoing research into the mechanisms of skin aging and the development of treatments.

Over the past decade, the cosmetic and pharmaceutical industries have introduced numerous products and techniques to mitigate the visible signs of skin aging. Innovations include the development of novel active ingredients, transdermal delivery (TDD) systems for enhanced penetration of actives, and advances in cosmetic surgical procedures. In the present invention, the inventors tried to find a solution that copies all positive effects of retinoid without the negative side effects that arise from enhanced stability, non-cytotoxicity and efficacy.

The primary focus was on the skin barrier enhancement potential, which appears to be responsible for a range of beneficial effects, including anti-inflammatory responses, epidermal cell growth and differentiation, increased collagen production, improved sebaceous gland function, reversal of photo-aging, inhibition of melanogenesis, and promotion of the extracellular matrix (ECM).

However, many effective active ingredients face challenges in compatibility, solubility, or maintaining stability within commonly accepted cosmetic carriers (solvents). These actives often separate, precipitate, or crystallize, leading to a significant reduction in their efficacy due to poor bioavailability and rapid degradation. The main objective of the present invention is to offer a practical solution that stabilizes HPR, enhances its overall efficacy, improves bioavailability, reduces toxicity, and delivers active ingredients in a cost-effective and safe manner. The compositions according to the invention are water dispersible complexes which comprise or consist of hydroxypinacolone retinoate (HPR) at 0.01% to 25% by weight, preferably at 1-10% and a cyclodextrin solid delivery agent which is preferably gamma (v)-cyclodextrin at 80-98% by weight, preferably 85-95%. The compositions can further include an antioxidant such as the water-soluble antioxidant alpha-glucosyl rutin at 1-10% by weight, preferably 4-6%. The sum content of hydroxypinacolone retinoate, gamma-cyclodextrin and alpha-glucosyl rutin will, of course, not exceed 100 wt. %.

Retinoic acid esters, such as, hydroxypinacolone retinoate (HPR), a non-prescriptive topical retinoid have been used for the treatment of skin aging (extrinsic and intrinsic factors). Hydroxypinacolone retinoate (I) is a light-yellow powder with limited solubility in both cosmetic and pharmaceutical formulations, and it exhibits poor stability as a pure powder. To address these formulation challenges, hydroxypinacolone retinoate (HPR or known as (I)) is shown below as Formula I:

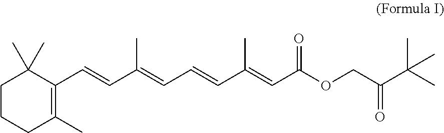 [SMILES: CC1=C(/C=C/C(C)=C/C=C/C(C)=C/C(=O)OCC(=O)C(C)(C)C)C(C)(C)CCC1]

has been dissolved in dimethyl isosorbide (DMI), which is sold commercially as Granactive Retinoid™ (Grant Industries, Inc.) or dissolved in diisopropyl adipate, (commercially known as Symrenew HPR (Symrise)). DMI improves both solubility and stability; however, this composition still faces challenges, as it needs to be stored in an inert atmosphere to maintain stability. This clear yellow liquid mixture allows for ease of use in the addition to various formulations (such as oil-in-water; water-in-oil; anhydrous). Additionally, formulating this composition into higher water content emulsion formulations presents further difficulties, i.e. precipitation of the HPR. Retinoids are lipophilic compounds and typically precipitate or crystallize out of formulations with high water content (>60%). In addition, the use of nitrogen for storage and application is highly recommended to prevent oxidative degradation.

The inventive compositions may also include, but is not limited to, peptides, growth factors, moisturizers, or auxiliary antioxidants which, when combined with HPR, enhance the overall efficacy of the composition. This enhancement may result from either the improved stability of the active ingredient (HPR) or from synergistic effects with other bioactive components. Excipients may include carriers, stabilizers, emulsifiers, or solvents, well known to those of ordinary skill in the art which support the stability and application of the composition.

These compositions may be formulated into various topical preparations, including creams, gels, lotions, serums, or ointments. The concentration of HPR may range from 0.01% to 5%, depending on the desired strength and specific application.

The present invention also provides topical compositions comprising a combination of hydroxypinacolone retinoate (HPR) cyclodextrin complex, a bioactive, and suitable excipients. These compositions are preferably topical preparations, more specifically pharmaceutical or cosmetic formulations, with a particular focus on cosmetic applications. It has been discovered that these combinations of retinoids are effective in preventing or treating age- and stress-related signs of skin aging.

### Cyclodextrin as a Delivery Excipient

Delivery of the active into the skin is necessary to promote activity. Without being bound to theory, only a micromolar amount of the active topically applied is necessary to activate the Retinoic Acid Receptor Element (RARE). Once topically applied, the active must permeate the skin, into the cells, and eventually cross into the nucleus to bind to the Retinoic acid receptor to allow for the transcriptional process to occur. Dimethyl isosorbide is a permeation enhancer and has been reported to dissolve retinoids, more specifically hydroxypinacolone retinoate. Dimethyl isosorbide has been reviewed as a topically safe ingredient and used in pharmaceutical applications.

The present invention also provides topical dermal compositions that include microsphere-encapsulated retinoids. However, hydroxypinacolone retinoate (HPR) is poorly soluble in aqueous systems and is highly sensitive to light and air, leading to rapid degradation. These challenges can be addressed through encapsulation into cyclodextrins (CDs), which are non-toxic cyclic oligosaccharides derived from the enzymatic conversion of starch. Cyclodextrins, composed of cyclic oligomers linked by α-1,4 glycosidic bonds, are characterized by a truncated cone structure, featuring a hydrophilic outer surface and a hydrophobic cavity. The 3 most common CDs are alpha-cyclodextrin (α-CD), beta-cyclodextrin (β-CD) and gamma-cyclodextrin (γ-CD) as shown below in Formula II:

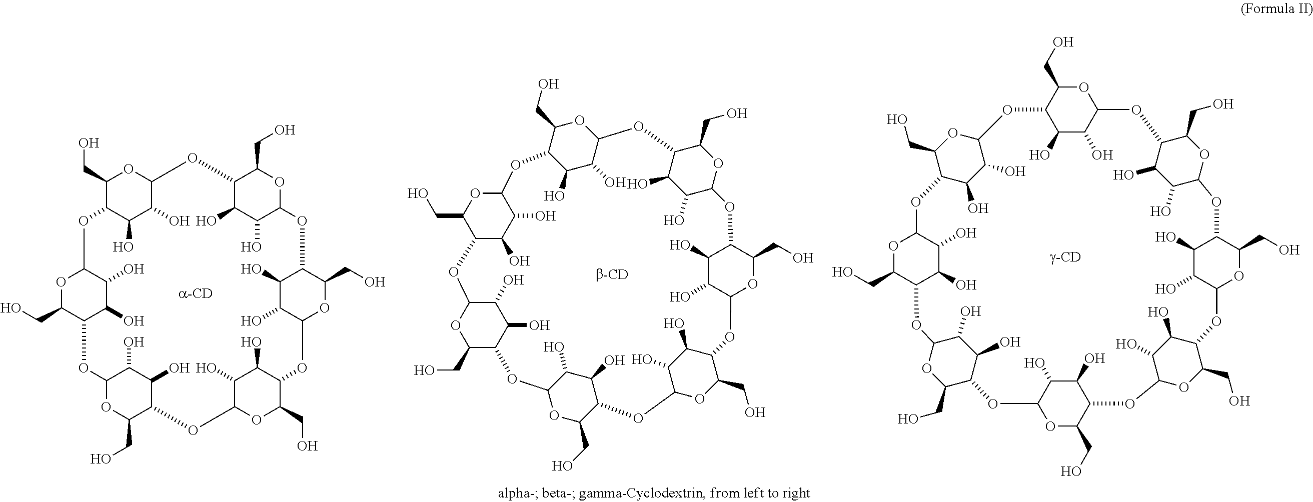

Each of the cyclodextrins differ in the number of glucose units, with six (6) for α-CD, seven (7) for β-CD and eight (8) for γ-CD. With increasing number of glucose units, the hydrophobic cavity of the CD increases which allows a wider range of molecules with different properties (hydrophobic, lipophobic and hydrophilic) to complex with the CDs. For example, an illustration of gamma-cyclodextrin with the bucket-shape typical of the cyclodextrins as well as their hydrophobic core (gray) and hydrophilic exterior (white) are shown below in Formula (III):

In addition, the different glucose units can affect the solubility behavior of the final host-guest complex. This unique structure allows CDs to function as “all-purpose molecular containers” capable of selectively accommodating a wide range of molecules through supramolecular host-guest interactions, resulting in the formation of inclusion complexes (ICs). Cyclodextrins are chemically versatile and can be modified into mono- or poly-substituted derivatives to improve properties such as solubility, stability, and complexation abilities. The complexation process leads to significant changes in the guest molecules' spectral properties, reactivity, volatility, solubility, cytotoxicity, and stability. For the purposes of the present invention, encapsulation as used to describe the complexation of the CD with HPR shall be understood to include not only complete envelopment but also substantially complete or partial encapsulation of the HPR due to hydrogen bonding, Van der Waals forces and similar non-covalent interactions between HPR and CD.

Cyclodextrins have found significant applications in drug delivery and pharmaceutical technologies. Their most acknowledged use is in enhancing the aqueous solubility of poorly soluble drugs through the formation of CD/drug inclusion complexes. Moreover, cyclodextrins offer protection to drugs from heat, light, hydrolysis, and oxidation, thereby improving the stability of formulations. In certain applications, cyclodextrins can manipulate volatile compounds, mask unpleasant tastes and odors, and reduce the irritation caused by certain compounds. Additionally, cyclodextrins can modify the release rate of drugs, functioning as excipients for either immediate or sustained release. Both native and modified cyclodextrins are FDA-approved for pharmaceutical use and have been designated as a “generally recognized as safe (GRAS)” substances, and their widespread success is evidenced by the fact that over 50 medications currently on the market contain cyclodextrins. The increasing number of approved formulations suggests that cyclodextrins remain a valuable tool in the pharmaceutical industry, with expanding applications and promising prospects.

The process of encapsulation of HPR in cyclodextrins compared to non-complexed HPR can significantly increase its aqueous dispersibility and formulation compatibility and reduce its photodegradation rates while ensuring a controlled and gradual release. Due to the structure of the CDs, the complexation of hydrophobic molecules occurs by hydrophobic Van der Waals forces between the internal cavity of the CD and the guest compound(s). The use of forming complexes to enhance water dispersibility and/or solubility has been taught for many years in the food, fragrance, flavors, cosmetic and pharmaceutical industries. The method of processing has remained unchanged requiring a slurry and paste kneading method followed by a slow drying process. Other processes have been applied to make cyclodextrin complexes but make use of supercritical fluids (i.e. Carbon dioxide), although the implementation of supercritical fluids requires a steep investment in equipment and extensive training. This would not be ideal for most applications due to cost and revision of infrastructure. Mixing and drying in a mixer/blending vessel that is oriented vertical (i.e. jacketed conical screw mixer) or horizontal (i.e. jacketed plow mixer), where it is possible to create the encapsulated product. The mixing must be efficient enough to break apart agglomerates and not stick to the walls of the vessel. The motor being used to mix must have enough torque to handle the mixing of the slurry paste as it may undergo a dilatant phase. The mixing may proceed through a non-Newtonian phase that causes the slurry to become shear thickening as the ratio of liquid to powder decreases during the drying process. The vessel must be jacketed to allow heat to volatilize the fluids and connected to a distillation receiver for the fluids. It would be preferable to reduce the vacuum pressure of the system to depress the boiling points of the volatile fluids and minimize degradation of the active from exposure to excessive heat.

Additional drying methods may be necessary to achieve a dryness of >85% and water activity of <0.60 (a/w). Obtaining a water activity of <0.60 greatly reduces microbial growth by preventing the availability of water needed for microbial proliferation. The water activity of a material is the ratio between the vapor pressure of the material itself, when in a completely undisturbed balance with the surrounding air media, and the vapor pressure of distilled water under identical conditions. Additional drying methods may include but are not limited to convection oven, vacuum oven, air drying with convection blower, jacketed drying vessel while mixing, jacketed drying vessel under vacuum while mixing, fluidized air bed drying, flash drying, spray drying, etc.

To obtain an ultra-fine powder for cosmetic applications, it would be desirable to post mill the granulated coarse powder followed by additional sieving to screen for mechanical defects. Milling processes may include hammermills, pin mill grinder, high speed blender, ball mill, jet mill, etc.

Utilizing the compositions and methods described in the present invention, the retinoid can be effectively delivered deep into the skin, where it can interact with retinoid receptors.

The present invention provides a cosmetic composition for making up and/or caring for the skin comprising a compound of HPR complexed with gamma-cyclodextrin, a cosmetically acceptable excipient. While not wishing to be limited by theory, it is believed that HPR becomes encapsulated by (i.e., complexed within) the hydrophobic core of gamma-cyclodextrin as illustrated below in Formula IV:

### (Formula IV) Illustration of Host-Guest Complexation of HPR and Gamma-Cyclodextrin

The HPR and gamma-cyclodextrin complex has been found to have excellent shelf-life stability without the presence of inert gas under ambient conditions unlike other retinoids stored as a fluid.

A “cosmetically acceptable excipient” means an excipient that is useful in preparing a cosmetic composition that is generally safe, non-toxic and neither biologically nor otherwise undesirable.

The present invention also encompasses a topical dermal composition comprising a plurality of biodegradable polymeric microparticle, mineral or liposomal, each containing a retinoid. The microparticles are dispersed in a vehicle consisting of both aqueous and non-aqueous solvents. The biodegradable polymers used to create the microparticles may include polymeric lactic acid, polymeric glycolic acid, polymeric lactic acid glycolic acid (PLGA), Saccharomyces Ferment, lauroyl lysine, polyhydroxyalkanoates (PHA), cellulose, starch or combinations thereof. Examples of mineral microparticles include but are not limited to hydroxyapatite, mica, zeolite, etc. Liposomes are made of lipids, fatty acids, phospholipids, polysaccharides, surfactants, polyethylene glycol, etc. and mixtures thereof. Loading of actives, such as retinoids within the encapsulation unit(s) can range from 0.01% to 25%, depending on the desired formulation.

An antioxidant can further protect the retinoid from damage caused by free radicals. Due to the HPR's structure, characterized by a chain of conjugated double bonds, it makes it susceptible to interaction with free radicals, leading to degradation and loss of biological activity. Free radicals are produced as byproducts of normal cellular metabolism and can be generated by external factors, such as photoirradiation, hydroxyl and peroxyl, and environmental pollutants. Aside from antioxidation properties, the antioxidant may prevent metabolic enzymes from the degradation of retinoids. It is widely known that cytochrome P450 (CYP) enzymes, including CYP26 and CYP27C1, are involved in the degradation of retinoids and play a crucial role in metabolizing both endogenous and exogenous substances, influencing drug efficacy, toxicity, and skin health, and are therefore important in drug development for skin diseases. Antioxidants can include but are not limited to flavonoids, such as quercetin, rutin, alpha-glucosyl rutin, glutathione, tocopherols (Vitamin E), ascorbic acid (Vitamin C), butylated hydroxyanisole (BHA), butylated hydroxytoluene (BHT), uric acid, ubiquinol, etc.

A preferred antioxidant used in the invention are flavonoids such as but not limited to alpha-glucosyl rutin (AGR) represented by Formula (V) and quercetin of represented by Formula (VI) as shown below:

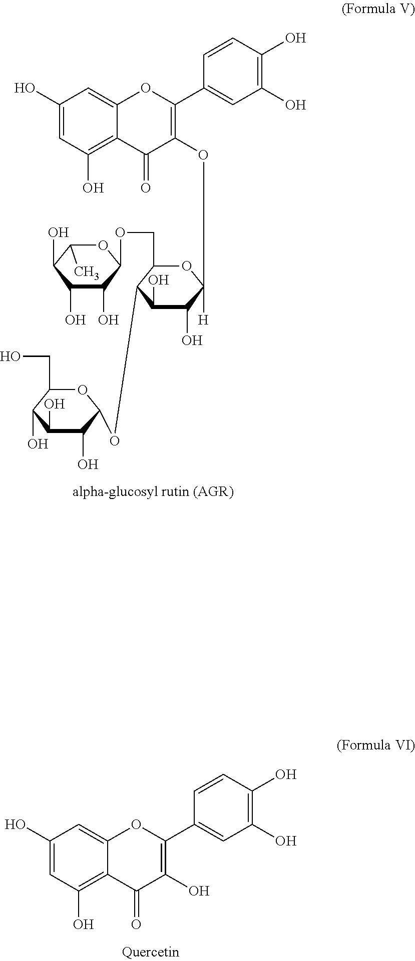

Depending on the chemical structure of flavonoids, they can be further characterized into subgroups. Examples of flavonoids that belong to each subgroup
Flavanone—Hesperetin; Naringenin; Eriodictyol
Flavone—Apigenin; Chrysin; Luteolin; Baicalein; Tangeretin
Flavonol—Kaempferol; Quercetin; Myricetin; Rutin
Isoflavone—Genistein; Daidzein; Glycitein
Anthocyanin—Cyanidin; Malvidin; Petunidin
Chalcone—Arbutin; Phloretin; Phlioridzin.

In accordance with the invention, it is preferrable to have antioxidants with different solubility parameters. For example, AGR (V) is known to be a water-soluble flavonoid while quercetin (VI) is soluble in anhydrous fluids, such as, dimethyl isosorbide, oils, emollient esters, etc.

However, due to Quercetin coming from natural origins, its quality and purity is not guaranteed. Steps are required to obtain a refined product with enhanced performance and quality.

It is common to enzymatically functionalize flavonoids with glucosyl groups to enhance bioavailability by increasing water solubility. By increasing the water solubility, such as with AGR (V), the flavonoid can quench ROS between extracellular membranes. Alternatively, by various means of membrane transport, more specifically called Active Transport, hydrophilic molecules bind to carrier protein and are delivered into the cell. Once inside the cell, enzymes known as Glycosylases can hydrolyze (Scheme 1) the alpha-glucosyl group to entrap the hydrophobic rutin, convert it to quercetin, and quench ROS within the cells. The glucosyl group can be converted to energy for other cellular processes.

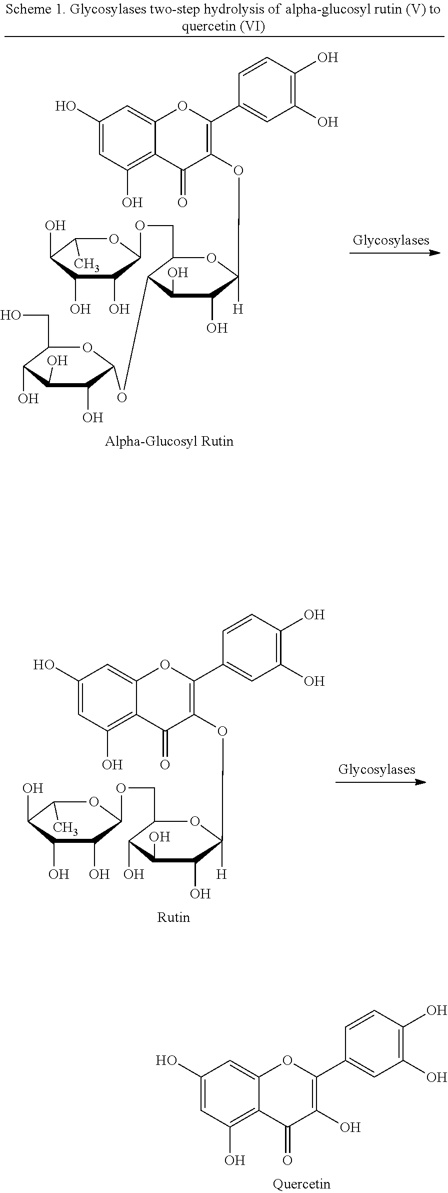

Furthermore, the present invention also provides a cosmetic composition for making up and/or caring for the skin and/or lips comprising a water dispersible complex of HPR/gamma-CD (as illustrated by Formula IV) and a cosmetically acceptable excipient, preferably alpha-glucosyl rutin (AGR) (Formula V). This composition can be added to the hydrophilic phase or hydrophobic phase of a formulation, but preferably to the aqueous phase. The invention also discloses the inclusion of quercetin (Formula VI) in the oil miscible composition of HPR (I) and dimethyl isosorbide.

### Bioactive Enhancer

Herein, a “bioactive enhancer” is an active that can enhance synergistic skin care efficacy of HPR in preparing a cosmetic composition derived from natural sources or synthetic sources. In addition, HPR in dimethyl isosorbide was compatible and stable with another “bioactive enhancer”, known as, bakuchiyl salicylate (VII) in glyceryl linoleate and glyceryl linolenate. The structure for bakuchiyl salicylate is shown below as Formula VII:

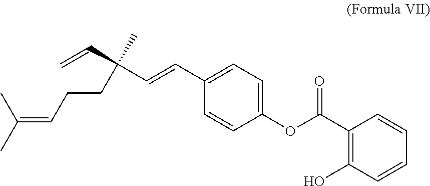 [SMILES: C=C[C@@](C)(/C=C/c1ccc(OC(=O)c2ccccc2O)cc1)CCC=C(C)C]

The combination of both HPR and Bakuchiol Salicylate (VII) is beneficial for enhanced skin care activity.

Another aspect of the invention is to allow the extension of lipophilicity and compatibility compared to bakuchiol that enhances superior skin permeation over the parent compound bakuchiol. Compatibility allows better permeation of the active into the skin, as well as better stability in the formulation to prevent degradation and prevent separation from the composition of the formulation. A goal of the invention is to improve and enhance the compatibility and stability to prevent separation from the formulation which would lead to haze, precipitation, crystallization, destabilization, discoloration and appearance of the formula. The compositions of the invention are therefore unlike prior art compositions which have poor compatibility and stability, and which lead to poor skin care performance because the active does not penetrate the skin effectively.

The slow delivery and more uniform delivery of an active by penetration into the skin is highly beneficial for treating sensitive skin, skin ageing and skin disorders. A time-release and uniform delivery is achieved by enhancing lipophilic properties of the active to enhance formulation compatibility and by enhancing molecular properties to allow skin penetration followed by hydrolysis of the active. Bakuchiyl Salicylate (VII) has been shown to demonstrate this behavior.

### Definitions of Molecules

The molecules, compounds, and mixes thereof referenced below are used interchangeably within this document, wherein:
1. Hydroxypinacolone retinoate is (Formula I), HPR.
2. Gamma-cyclodextrin is g-cyclodextrin, gamma-CD, g-CD, gCD, CD, γ-cyclodextrin, γ-CD, γCD, cyclodextrin, (Formula III).
3. Alpha-glucosyl rutin is (Formula V), alpha-glucosylrutin, α-glucosyl rutin, α-glucosylrutin, AGR, alpha-GR, α-GR, glucosylrutin, glucosyl rutin.
4. Quercetin is (Formula VI).
5. Bakuchiyl salicylate is (Formula VII), bakuchiol salicylate, Comp-1.
6. Retinoic acid receptor-alpha is retinoic acid receptor-α, RAR-alpha, RAR-α, RARa.
7. Gamma-cyclodextrin/hydroxypinacolone retinoate and encapsulated mixtures thereof as denoted in definitions 1 and 2 are terms for the complex of both molecules, also referenced as: encapsulated, encapsulation, complex, complexed, host-guest, host-guest complex, host-guest encapsulation, (Formula IV), CD/HPR, HPR/CD.
8. Gamma-cyclodextrin/hydroxypinacolone retinoate/alpha-glucosyl rutin and mixtures thereof as denoted in definitions 1, 2, and 3 are terms for the complex of definition 7 with the addition of alpha-glucosyl rutin, as defined in definition 3, also called as HPR/CD/AGR, CD/HPR/AGR.

In another aspect of this invention disclose a novel composition of one or more bio-active enhancers which act as complementary to each other and produce significant synergistic effect. Within this aspect of the invention, HPR (Formula I) complexed within a cyclodextrin can be formulated with one or more bioactive enhancers selected from bakuchiol esters. Suitable bakuchiol esters can be selected from a wide range of ester derivatives of bakuchiol (Formula VIII). For example, in some aspects, the bakuchiol ester can include bakuchiyl salicylate (Comp-1) but not limited to other ester derivatives such as bakuchiyl nicotinate (Comp-2), bakuchiyl isonicotinate (Comp-3), bakuchiyl glycolate (Comp-4), bakuchiyl mandeliate (Comp-5), bakuchiyl biotenate (comp-6), bakuchiyl quinolinate (Comp-7), and bakuchiyl salicyl nicotinate (Comp-8).

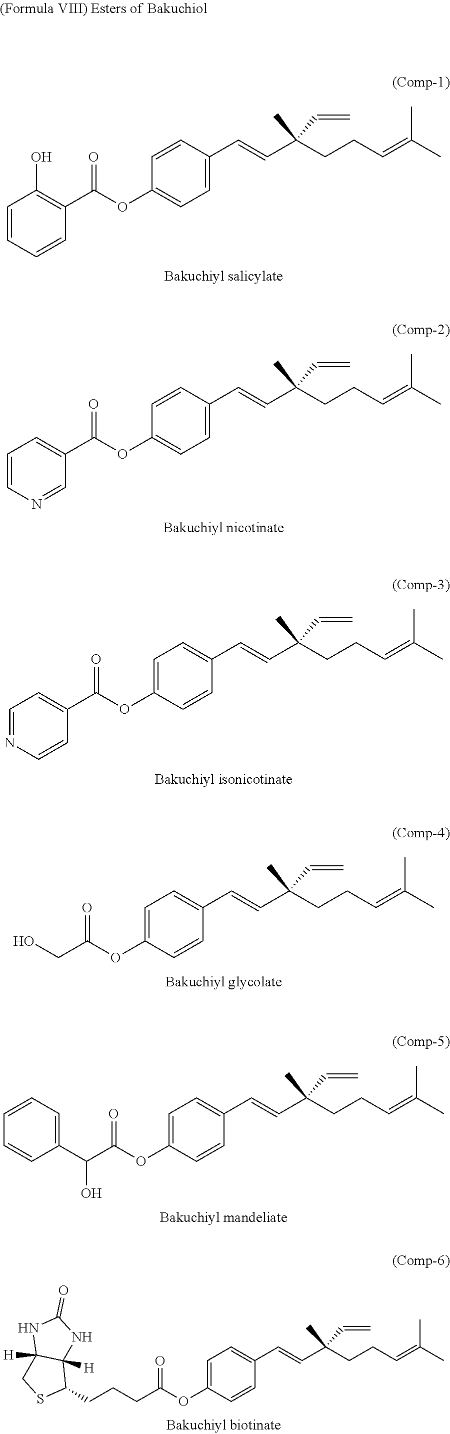 [SMILES: C=C[C@@](C)(/C=C/c1ccc(OC(=O)c2ccccc2O)cc1)CCC=C(C)C ; C=C[C@@](C)(/C=C/c1ccc(OC(=O)c2cccnc2)cc1)CCC=C(C)C ; C=C[C@@](C)(/C=C/c1ccc(OC(=O)c2ccncc2)cc1)CCC=C(C)C ; C=C[C@@](C)(/C=C/c1ccc(OC(=O)CO)cc1)CCC=C(C)C ; C=C[C@@](C)(/C=C/c1ccc(OC(=O)C(O)c2ccccc2)cc1)CCC=C(C)C ; C=C[C@@](C)(/C=C/c1ccc(OC(=O)CCC[C@@H]2SC[C@@H]3NC(=O)N[C@@H]32)cc1)CCC=C(C)C]

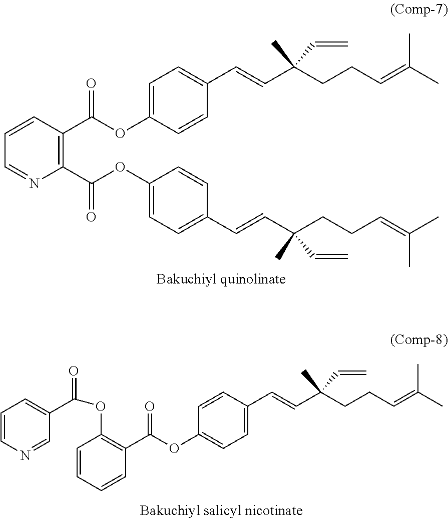 [SMILES: C=C[C@@](C)(/C=C/c1ccc(OC(=O)c2cccnc2C(=O)Oc2ccc(/C=C/[C@@](C)(C=C)CCC=C(C)C)cc2)cc1)CCC=C(C)C ; C=C[C@@](C)(/C=C/c1ccc(OC(=O)c2ccccc2OC(=O)c2cccnc2)cc1)CCC=C(C)C]

Anti-aging is a complex and multifaceted challenge, requiring a multi-target approach to effectively mitigate its effects. Relying on a single ingredient is insufficient to meet all the diverse needs of the skin as it ages. Thus, there is a continual demand for novel compositions that offer superior efficacy, compatibility with formulations, and favorable safety profiles.

Bakuchiol has gained significant attention in the skincare industry, hailed as a “natural alternative to retinol”. This compound, a meroterpene phenol isolated from the seeds of Psoralea Corylifolia, has been a cornerstone of Ayurvedic and Traditional Chinese Medicine for centuries. However, its use in cosmetics was limited until a natural, phototoxicity-free form of bakuchiol was developed, free from two phototoxic compounds: psoralen and iso-psoralen. Studies have shown that bakuchiol acts as a functional analog of retinol, as it demonstrates similar gene expression patterns to retinol. However, it does exhibit cytotoxicity at higher concentrations, with a narrow therapeutic window, which can limit its utility.

The present invention successfully addresses these issues by utilizing bakuchiol in a more effective and skin-friendly manner, offering several notable benefits.

One of the key components of this novel composition is ester derivative of bakuchiol, i.e. bakuchiyl ester. As an example, bakuchiyl salicylate is generated through esterification, where bakuchiol and salicylic acid are combined by synthetic methods known to those in the art. This bioactive ester offers several distinct advantages over bakuchiol alone.

Improved Skin Permeation: Bakuchiyl salicylate has superior skin penetration properties compared to both bakuchiol and salicylic acid. This increased bioavailability ensures that the active ingredients reach deeper layers of the skin, enhancing their overall effectiveness. Additionally, as the compound moves through different layers of the skin, natural esterases in the skin slowly release the active ingredients in a controlled manner, optimizing their therapeutic benefits while minimizing potential adverse effects.

Wider Therapeutic Index: While bakuchiol itself can be less tolerable at higher concentrations due to its narrow therapeutic index, bakuchiyl salicylate is much more tolerable and has a wider therapeutic index. This makes it a safer and more effective option for long-term use in skincare formulations.

Phototoxicity-Free: Naturally derived bakuchiol can contain trace amounts of phototoxic compounds such as psoralen and iso-psoralen, depending on the quality of the material. In contrast, Bakuchiyl salicylate is free from these harmful compounds, making it a safer choice for skincare applications, particularly in formulations intended for sun exposure.

Bakuchiyl salicylate (Comp-1) (CAS #3024002-49-8) is a unique bioactive which possesses several advantages over bakuchiol (SM-1) in terms of tolerability and efficacy. See U.S. Pat. No. 10,471,035, Krzysztof Bojanowski, the disclosure of which is incorporated herein by reference.

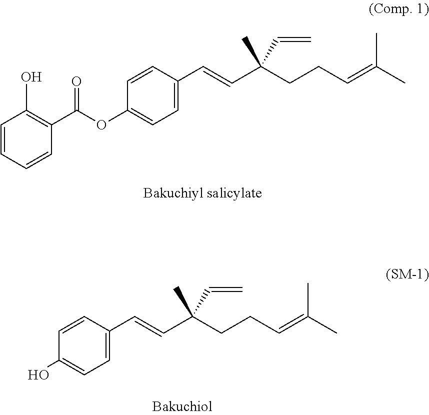 [SMILES: C=C[C@@](C)(/C=C/c1ccc(OC(=O)c2ccccc2O)cc1)CCC=C(C)C ; C=C[C@@](C)(/C=C/c1ccc(O)cc1)CCC=C(C)C]

### (Formula IX) Chemical Structures of Bakuchiyl Salicylate and Bakuchiol (SM-1)

Bakuchiyl salicylate is more lipophilic compared to bakuchiol and has superior skin permeation over the parent compound bakuchiol. As an ionic acid, salicylic acid typically stays on the surface of the skin epidermis and does not penetrate well into the dermis. It is a monographed anti-acne active ingredient in the US and other regions OTC applications. Besides acting on skin as its own active ingredient, (Formula VII) also has the potential to deliver salicylic acid via enzymatic hydrolysis into the skin dermis, thereby acting as a controlled release agent for salicylate, offering a more effective route for flawless skin. This innovative pro-coupling of actives could deliver the dual benefits of bakuchiol and salicylic acid, along with its precursors, to the skin. Salicylic acid could enhance the exfoliating effect and act as a strong anti-microbial (C. acnes) agent. Furthermore, salicylic acid exhibits significant antimicrobial activity, particularly against Cutibacterium acnes (formerly Propionibacterium acnes), the bacterium responsible for acne vulgaris, thereby enhancing the formulation's ability to address acne-related concerns.

Additionally, bakuchiol has been shown to have anti-aging effects similar to retinol, further complementing the benefits of niacin in promoting youthful, healthy skin.

Another aspect of the invention is to allow the extension of lipophilicity and compatibility compared to bakuchiol that enhances superior skin permeation over the parent compound bakuchiol. Compatibility allows better permeation of the active into the skin, as well as better stability in the formulation to prevent degradation and prevent separation from the composition of the formulation. A key goal of the invention is to improve and enhance the compatibility and stability to prevent separation from the formula which would lead to haze, precipitation, crystallization, destabilization, discoloration and appearance of the formula. The compositions of the invention are therefore unlike prior art compositions which have poor compatibility and stability, and which lead to poor skin care performance because the active does not penetrate the skin effectively.

The slow delivery and more uniform delivery of an active by penetration into the skin is highly beneficial for treating sensitive skin, skin ageing and skin disorders. A time-release and uniform delivery is achieved by enhancing lipophilic properties of the active to enhance formulation compatibility and by enhancing molecular properties to allow skin penetration followed by hydrolysis of the active. Bakuchiyl Salicylate (Comp-1) has been shown to demonstrate this behavior.

### Biological Assays

The present invention also provides a topical composition as described hereinabove and further comprising bakuchiol ester derivatives for skin treatments. The preferred derivatives included with the HPR cyclodextrin complexes are represented by the following compounds: Comp-1, Comp-2, Comp-3, Comp-4, Comp-5, Comp-6, Comp-7, and Comp-8. These compounds have been synthesized and tested for their effects on key biomarkers and cellular pathways involved in the aging process with all compounds having a potential for a high level of activity.

Most of these derivatives have been evaluated using various biological assays, including but not limited to collagen synthesis, elastin synthesis, hyaluronic acid production, and modulation of key transcription factors such as NRF2 and NF-kB. Notably, Comp-1, Comp-2, Comp-7 and Comp-8 demonstrated top-tier activity in PCR type gene expression assays, making them particularly useful as active ingredients in anti-aging cosmetic formulations.

The anti-aging properties of these bakuchiol ester derivatives were evaluated through a series of biological assays, including:

Collagen Synthesis: The ability of the derivatives to stimulate collagen production was assessed by measuring collagen content in cultured skin cells.

Elastin Synthesis: The derivatives were tested for their ability to enhance elastin production, which is critical for maintaining skin elasticity.

Hyaluronic Acid Production: The production of hyaluronic acid, a major component of the skin extracellular matrix, was measured to evaluate the potential of the derivatives in improving skin hydration.

Nuclear Factor Erythroid 2-Related Factor 2 (NRF2) Activation: NRF2 is a key transcription factor involved in the regulation of cellular stress responses. The activity of the derivatives in activating NRF2 was assessed to determine their potential as antioxidant agents.

Nuclear Factor Kappa-B (NF-kB) Inhibition: NF-kB is a transcription factor involved in inflammation. The ability of the bakuchiol ester derivatives to inhibit NF-kB signaling was measured to evaluate their anti-inflammatory effects.

Among the tested compounds, Comp-1, Comp-7 and Comp-8 demonstrated particularly strong effects in one or more of all the biological assays, indicating their superior anti-aging activity. These compounds were found to significantly enhance collagen and elastin production, stimulate hyaluronic acid synthesis, activate NRF2, and inhibit NF-KB, all of which are important for promoting skin health and reducing the signs of aging.

### Challenges in Skin Penetration and Transdermal Delivery:

One of the key challenges faced by nicotinic acid and niacinamide in topical formulations is their poor penetration across the skin's phospholipid bilayer, particularly the stratum corneum. This limits their effectiveness in traditional skincare products. However, advancements in transdermal delivery (TDD) technology have provided a solution to this challenge.

By enhancing the permeability of active ingredients through the stratum corneum, TDD technology enables the controlled and passive delivery of crucial actives such as nicotinic acid and quinolinic acid directly into the epidermis. This approach has gained significant traction in the anti-aging market, as it allows for more effective delivery of key nutrients and compounds to the skin.

One promising development in transdermal delivery is the formation of its ester using lipophilic alcohol, e.g. bakuchiol nicotinate (Comp-2), bakuchiol quinolinate (Comp-7) and bakuchiol salicylnicotinate. As an ester of nicotinic acid/or quinolinic acid and bakuchiol, this compound is expected to exhibit superior skin permeability compared to its parent molecules. Upon topical application, these ester derivatives would pass through the stratum corneum and into the epidermis, where it would be hydrolyzed by esterases, particularly abundant in the epidermis, to release both bakuchiol and corresponding acids.

This innovative coupling of actives deliver the dual benefits of bakuchiol and niacin and its precursors to the skin, enhancing the NAD+ pool, improving mitochondrial function, reducing oxidative stress, and supporting skin barrier integrity. Additionally, bakuchiol has been shown to have anti-aging effects similar to retinol, further complementing the benefits of niacin in promoting youthful, healthy skin.

The combination of HPR complexed within a cyclodextrin and an ester derivative of bakuchiol produces a synergistic effect, wherein the individual benefits of each ingredient are amplified. Retinoids exert their effects through transcription factors by activating nuclear receptors, which regulate cellular differentiation, proliferation, and collagen synthesis. In contrast, the ester derivative of bakuchiol operates at a molecular level, likely by modulating the activity of specific signaling pathways involved in skin regeneration and anti-inflammatory responses. Additionally, niacin (vitamin B3) contributes to this formulation by improving the skin's barrier function, reducing trans-epidermal water loss (TEWL), and enhancing skin hydration. Niacin also exhibits anti-inflammatory properties, which can help to alleviate redness and irritation, particularly when used in conjunction with other actives like retinoids. Alternatively, salicylic acid can have better penetration into the skin as a bakuchiol ester derivative and undergo hydrolysis, whereby the salicylic acid may provide anti-microbial benefits (i.e. acne treatment). This novel composition offers a “triple-action” approach by simultaneously delivering three active components-HPR and bioactive enhancers which have functional groups that allow hydrolysis to different compounds that are known to poorly penetrate the skin and then allow each compound to target distinctive mechanisms of skin rejuvenation and enhanced activity of HPR. Notably, the formulation demonstrates a significantly reduced potential for irritation compared to traditional retinoid-based compositions, making it a more suitable option for individuals with sensitive skin. This enhanced tolerance profile is particularly beneficial for those seeking the anti-aging, anti-inflammaging, acne-fighting, and barrier-repairing benefits of retinoids, bakuchiol, salicylic acid and niacin without the typical side effects of dryness, peeling, or redness.

### Results Section

### Activation of Retinoic Acid Receptor Element (RARE)

The Retinoic Acid Receptor (RAR) forms heterodimers with the Retinoid X Receptor (RXR) to initiate DNA transcription that leads to a cascade, resulting in skin rejuvenation. The activation of this receptor elucidates hundreds of regulatory genes and pathways. The RAR does not form dimers to itself. The activation of the RXR alone is not enough to promote the transcription process. The binding of Retinoic acid and analogs to the RAR initiates a response of the transcription factors, which when coupled with a luciferase reporter gene, is able to be quantified in an assay with a luminometer. This concept is referred to as a Retinoic Acid Response Element (RARE) assay. RARE assays were used to quantify the activation of the RAR by HPR. Therefore, this RARE assay would provide strong evidence that the retinoid, HPR (I) is able to exit from the host-guest complex and bind specifically to the nuclear receptor. The specific receptor chosen for the RARE assay was the RAR-alpha.

HPR complexed within gamma-cyclodextrin and mixed with alpha-glucosyl rutin (AGR) demonstrates that HPR is able to exit the cyclodextrin complex and initiate transcription of RAR-alpha as confirmed by luciferase activity measured by the RARE test kit from Indigo (cat. #IB02201, lot #230519). Complexed HPR/gamma-cyclodextrin with AGR displayed two-times greater efficiency on the log scale in the RAR-α receptor than HPR dissolved in DMI. At 50% maximal response, a lower concentration (molarity) of HPR in gamma-cyclodextrin with AGR is achieved with the same luciferase activity as HPR in DMI. In summary, HPR was able to exit the gamma-cyclodextrin and have two-times more binding efficiency due to the presence of the AGR. AGR protected HPR from degradation by ROS and metabolic breakdown by CYP P450 enzymes.

In Table 1 below, the stability of the complex HPR/gamma-CD (IV) was evaluated by RARE assay (BPS Biosciences, RARa Luciferase Reporter HEK293 Cell Line, Catalog #60503) under accelerated conditions. Complex HPR/gamma-CD (IV) was subject to a 45° C. environment for a maximum of 12 weeks. The initial sample was frozen until testing, as were all time points, 2, 4, and 8 weeks until the time to conduct the RARE assay study. The stability samples of complex HPR/gamma-CD were found to be unexpectedly very stable. In comparison, HPR in DMI would expect to have 0% residual activity by 72 hours due to full thermal degradation and as a result no activity on the RARE assay. Water was tested as a control in which all sample articles were compared. Transcription within the same range was observed for all the samples at all time points, and consistent RARE activation was seen for concentrations of HPR (I) ranging from 5 to 50 μM. This RARE assay proves HPR is stable when encapsulated in gamma-cyclodextrin and able to leave the encapsulation and enter the nucleus of skin cells to initiate RAR-α and begin skin rejuvenation. In addition, no cytotoxic activity was found among the broad concentration range.

| Test | Weeks at | Test | RARE activity | |
|---|---|---|---|---|
| Material | 45° C. | Concentration | (% Control) | p value |
| H₂O | N/A | N/A | 100 | 1.00 |
| CD/HPR | 0 | 50 μM HPR | 200 | 0.00 |
| (95/5) | | 25 μM HPR | 202 | 0.00 |
| | | 5 μM HPR | 195 | 0.00 |
| | 2 | 50 μM HPR | 189 | 0.00 |
| | | 25 μM HPR | 210 | 0.00 |
| | | 5 μM HPR | 216 | 0.00 |
| | 4 | 50 μM HPR | 185 | 0.00 |
| | | 25 μM HPR | 203 | 0.00 |
| | | 5 μM HPR | 206 | 0.00 |
| | 8 | 50 μM HPR | 181 | 0.00 |
| | | 25 μM HPR | 201 | 0.00 |
| | | 5 μM HPR | 203 | 0.00 |
| | 12 | 50 μM HPR | 209 | 0.00 |
| | | 25 μM HPR | 196 | 0.00 |
| | | 5 μM HPR | 194 | 0.00 |

Table 1. RARE assay demonstrating accelerated stability study of Complex HPR/gamma-cyclodextrin (IV) at 45° C. for 12 weeks. Complexed HPR/gamma-CD maintained consistent activity of at least 97% when comparing 25 μm at 12 weeks to 0 weeks. Complexed HPR/gamma-CD remained stable, efficacious and non-cytotoxic.

In Table 2 below, the stability of HPR complexed within gamma-CD (IV) was evaluated by a RARE assay (BPS Bioscience RARa Luciferase Reporter HEK293 Cell Line, Catalog #60503) was used to determine the stability of different retinoids, namely hydroxypinacolone retinoate (HPR), retinol, retinaldehyde, and retinyl palmitate encapsulated in gamma-cyclodextrin (Example 2, Comparative Examples 18, 19, 20, respectively). All samples were made to have the same molar ratio of retinoid to CD and processed using the exact same preparation method. Samples were kept at 50° C. for four weeks and compared to their initial timepoints (freshly made compounds were frozen until tested). Each sample was then dispersed in water at 5, 25, and 50 μM retinoid concentrations and added to the cells to determine RARE activity. The data is shown as a percent of RARE activity, relative to pure water as a control. All-trans retinoic acid (ATRA) was used as a positive control in this study, validating the assay.

The RARE assay results demonstrate that encapsulating Hydroxypinacolone Retinoate (HPR) in gamma-cyclodextrin significantly enhances its stability, maintaining consistent efficacy even after rigorous accelerated aging tests of four weeks at 50° C. This complex not only ensures high thermal stability but also establishes a favorable balance between potency and safety. By evaluating cytotoxicity and a concentration range (5-50 μm), the data confirms that the encapsulated HPR performed unexpectedly superior over the other retinoids tested.

In contrast, retinol encapsulated in cyclodextrin was cytotoxic for the cells at 25 and 50 μM at the initial timepoint, but after being exposed to 50° C. for 4 weeks, the retinol degraded. The cytotoxicity of the retinol led to a decrease in RARE activity at the initial timepoint. After 4 weeks, the 25 and 50 μM samples fell within a tolerable range for the cells and it was concluded that the retinol had degraded to a level that was less cytotoxic by the cells. This phenomenon can be observed by the increase in RARE activity for those two concentrations (25 and 50 μM) after 4 weeks. Retinol was tolerated and not cytotoxic at 5 μM at the initial timepoint (0 week). The decrease in activity of the sample at 5 μM after 4 weeks exemplifies the degradation and poor stability of retinol.

Like retinol, retinaldehyde was also cytotoxic at 25 and 50 μM. Retinaldehyde did not degrade substantially at 50° C. after 4 weeks, and was determined to be cytotoxic at those concentrations. This is important to note because retinaldehyde is known to be the most unstable of the vitamin A family due to the unstable nature of the aldehyde functional group. This demonstrates that cyclodextrin may form an acetal complex and enhance the stability of the retinaldehyde (U.S. Pat. No. 8,410,079 B2).

Retinyl Palmitate was effectively protected from degradation by cyclodextrin encapsulation. However, it is less efficacious than HPR at the same concentrations. In fact, retinyl palmitate (5 μM) initiates less of a RARE response than the control, meaning that it is not initiating a strong transcription activity.

It is important to note that in this study the retinoids do not need to be converted into retinoic acid to display activity. HPR does not require biological conversions to bind effectively to the receptor.

TABLE 2
Comparison performance of retinoid complexed in gamma-cyclodextrin.

| | | | | | Cyto- | | RARE |
|---|---|---|---|---|---|---|---|
| | | Retinoid | | | toxicity | Degradation | Activation |
| | | Concen- | | | (CT!!) \| | (D [symbol] ) \| | Concen- |
| | Time | tration | % | p | Safety | Stability | tration |
| Test Material | (weeks) | (μM) | Control | value | (✓) | (✓) | Efficiency |
| H₂O | N/A | N/A | 100 | 1 | N/A | N/A | N/A |
| CD/HPR (95% wt./ | 0 | 50 | 170 | 0.000 | ✓ | ✓ | ✓ |
| 5% wt.) | | 25 | 173 | 0.000 | ✓ | ✓ | ✓ |
| | | 5 | 141 | 0.000 | ✓ | ✓ | ✓ |
| CD/HPR (95%/5%) | 4 | 50 | 157 | 0.000 | ✓ | ✓ | ✓ |
| 50° C. | | 25 | 161 | 0.000 | ✓ | ✓ | ✓ |
| | | 5 | 148 | 0.000 | ✓ | ✓ | ✓ |
| CD/Retinol | 0 | 50 | 107 | 0.000 | CT!! | ✓ | ✓ |
| (96.41%/3.59%) | | 25 | 125 | 0.000 | CT!! | ✓ | ✓ |
| | | 5 | 170 | 0.000 | ✓ | ✓ | ✓ |
| CD/Retinol | 4 | 50 | 164 | 0.000 | ✓ | D [symbol] | ✓ |
| (96.41%/3.59%) | | 25 | 172 | 0.000 | ✓ | D [symbol] | ✓ |
| 50° C. | | 5 | 137 | 0.000 | ✓ | D [symbol] | ✓ |
| CD/Retinaldehyde | 0 | 50 | 79 | 0.239 | CT!! | ✓ | ✓ |
| (96.43%/3.57%) | | 25 | 101 | 0.001 | CT!! | ✓ | ✓ |
| | | 5 | 163 | 0.000 | ✓ | ✓ | ✓ |
| CD/Retinaldehyde | 4 | 50 | 60 | 0.000 | CT!! | ✓ | ✓ |
| (96.43%/3.57%) | | 25 | 95 | 0.015 | CT!! | ✓ | ✓ |
| 50° C. | | 5 | 145 | 0.000 | ✓ | D [symbol] | ✓ |
| CD/Retinyl | 0 | 50 | 154 | 0.000 | ✓ | ✓ | X |
| Palmitate | | 25 | 134 | 0.000 | ✓ | ✓ | X |
| (93.42%/6.58%) | | 5 | 96 | 0.011 | ✓ | ✓ | X |
| CD/Retinyl | 4 | 50 | 155 | 0.000 | ✓ | ✓ | X |
| Palmitate | | 25 | 138 | 0.000 | ✓ | ✓ | X |
| (93.42%/6.58%) | | 5 | 81 | 0.544 | ✓ | ✓ | X |
| 50° C. | | | | | | | |
| ATRA | N/A | 1 | 160 | 0.000 | N/A | N/A | N/A |

Complex HPR/gamma-cyclodextrin maintained consistent activity under accelerated stability conditions of 50° C. for 4 weeks. All concentrations of CD/HPR meet the objective of consistent non-cytotoxicity, stability and efficacy. CD/Retinol and CD/Retinaldehyde at the concentration of 5 μm also met the objective. All other retinoid concentrations failed to meet the objectives. 15% difference to the initial timepoint is the degradation threshold.

### EXAMPLES

### Example 1: Method to Produce Refined Hydroxypinacolone Retinoate and Quercetin Fluid

Into a beaker with a mixer was added hydroxypinacolone retinoate (I) powder (10%) and Quercetin powder (5%) in dimethyl isosorbide (85%). The mixture was stirred until a homogeneous fluid was obtained. The fluid was slightly hazy and had a slight green-yellow-brown hue. The fluid was filtered through a 0.2 microns filter to obtain a clear amber fluid. The product is stored under the presence of inert atmosphere. The HPLC confirmed the presence of 10% HPR and 5% Quercetin.

### Example 2: Method to Produce Complex of Gamma-Cyclodextrin and Hydroxypinacolone Retinoate

In a beaker, HPR (I) (10%) was dispersed in isopropanol (90%) and heated to less than 80° C. until dissolved and the solution is clear yellow (Phase A). Gamma-Cyclodextrin (55%) was added to a standing KitchenAid™ mixer (Model No. KSM150PSMC) equipped with a flat beater and water (45%) was added and mixed (Phase B). Phase B can behave as a non-Newtonian fluid at concentrations of 40-70%. Phase A (23%) was added to Phase B (77%) and kneaded for 30 minutes to a dough-like consistency or slurry. During the mixing process of Phase A into Phase B, a dilatant phase (shear-thickening) is reached but is then surpassed as Phase A is continually added to Phase B. The slurry was scooped out of the mixer bowl and spread evenly onto oven safe trays. The trays were placed in an oven at 65° C. After about 16 hours, the powder cakes were tested for dryness by moisture balance and transferred to a mill to obtain a free-flowing powder. The powder is expected to have a loss of drying of less than 15%.

### Example 3: Method to Produce Complex of Gamma-Cyclodextrin and Hydroxypinacolone Retinoate with Alpha-Glucosyl Rutin

In a beaker, HPR (I) (10%) was dispersed in isopropanol (90%) and heated to less than 80° C. until dissolved and the solution is clear yellow (Phase A). Gamma-Cyclodextrin (55%) was added to a standing KitchenAid™ mixer (Model No. KSM150PSMC) equipped with a flat beater and water (45%) was added and mixed (Phase B). Phase B can behave as a non-Newtonian fluid at concentrations of 40-70%. Phase A (24%) was added to Phase B (76%) and kneaded for 30 minutes to a dough-like consistency or slurry. During the mixing process of Phase A into Phase B, a dilatant phase (shear-thickening) is reached but is then surpassed as Phase A is continually added to Phase B. The slurry was scooped out of the mixer bowl and spread evenly onto oven safe trays. The trays were placed in an oven at 65° C. After about 16 hours, the powder cakes were tested for dryness by moisture balance and transferred to a mill to obtain a free-flowing powder. The powder is expected to have a loss of drying of less than 15%. Alpha-glucosyl rutin (AGR) (CAS #130603-71-3) (5%) was added to the powder and mixed until homogenous.

### Example 4: Method to Produce Complex of Gamma-Cyclodextrin and Hydroxypinacolone Retinoate (23.5%)-Upper Limit Loading

In a beaker, Hydroxypinacolone retinoate (HPR) (15%) was dispersed in isopropanol (85%) and heated to less than 80° C. until dissolved and the solution is clear yellow (Phase A). Gamma-cyclodextrin (55%) was added to a standing KitchenAid™ mixer (Model No. KSM150PSMC) equipped with a flat beater and water (45%) was added and mixed (Phase B). Phase B can behave as a non-Newtonian fluid at concentrations of 40-70%. Phase A (53%) was added to Phase B (47%) and kneaded for 30 minutes to a dough-like consistency or slurry. During the mixing process of phase A into phase B, a dilatant phase (shear-thickening) is reached but is then surpassed as phase A continues to be poured. The slurry was scooped out of the mixer bowl and spread evenly onto oven safe trays. The trays were placed in an oven at 65° C. After about 16 hours, the powder cakes were tested for dryness by moisture balance and transferred to a mill to obtain a free-flowing powder. This example demonstrates that above this 23.5% HPR limit, the excess HPR does not encapsulate, and this excess was not stabilized.

### Example 5: Method to Produce Complex of Gamma-Cyclodextrin and Hydroxypinacolone Retinoate (0.018%)—Lower Limit Loading

In a beaker, Hydroxypinacolone retinoate (HPR) (1%) was dispersed in isopropanol (99%) and heated to less than 80° C. until dissolved and the solution is clear yellow (Phase A). Gamma-Cyclodextrin (55%) was added to a standing KitchenAid™ mixer (Model No. KSM150PSMC) equipped with a flat beater and water was added and mixed (Phase B). Phase B can behave as a non-Newtonian fluid at concentrations of 40-70%. Phase A (1%) was added to Phase B (99%) and kneaded for 30 minutes to a dough-like consistency or slurry. During the mixing process of phase A into phase B, a dilatant phase (shear-thickening) is reached but is then surpassed as phase A continues to be poured. The slurry was scooped out of the mixer bowl and spread evenly onto oven safe trays. The trays were placed in an oven at 65° C. After about 16 hours, the powder cakes were tested for dryness by moisture balance and transferred to a mill to obtain a free-flowing powder. This powder contained 0.018% HPR encapsulated in cyclodextrin

### Example 6: Method for HPLC Testing of Hydroxypinacolone Retinoate and Quercetin in DMI

The refined retinoid and quercetin fluid was diluted in methanol and injected into a C18, 3.5 μm, 4.6×10⁰ mm column on a reverse phase HPLC instrument. Using methanol as an eluent for a calibration curve, the concentration of HPR was determined. The diluted fluid was also injected into a C18-A, 5 μm, 4.6×250 mm column. Using a calibration curve with 50% acetonitrile and 50% water with 2% acetic acid, the concentration of quercetin was determined.

### Example 7: Method for Testing Complex of Hydroxypinacolone Retinoate and Gamma-Cyclodextrin

HPR (I) was extracted from the cyclodextrin by vigorous mixing in isopropanol for 10 minutes at 70 C. The solution was filtered and injected into a C18, 3.5 μm, 4.6×100 mm column on a reverse phase HPLC instrument. Using methanol as an eluent for a calibration curve, the concentration of HPR was determined. Fully complexed HPR had a recovery of about 65%. HPR encapsulation affinity with the gamma-CD was too high and difficult to extract 100% HPR. Isopropanol dissolves both gamma-cyclodextrin and HPR at higher temperatures, therefore it was chosen as the best extraction solvent for HPR. The extraction of 65% HPR resulted in high RARE activity.

### Example 8: Method for Testing Complex of Hydroxypinacolone Retinoate and Gamma-Cyclodextrin and Physical Blend

Non-encapsulated HPR was dissolved by vigorous mixing of powder in ethyl acetate for 10 minutes at 25 C. The solution was filtered and injected into a C18, 3.5 μm, 4.6×100 mm column on a reverse phase HPLC instrument. Using methanol as an eluent for a calibration curve, the concentration of HPR was determined. The CD/HPR powder had an HPR recovery of about 3%. The physical blend (comparative example 16) had an HPR recovery of 100%. Ethyl acetate does not dissolve gamma-cyclodextrin at all, but dissolves HPR very well. Therefore, it was chosen as a solvent to determine the amount of non-encapsulated HPR in the powder.

### Example 9: Qualitative Method for Determining Encapsulation Efficiency of Complex Gamma-Cyclodextrin and Hydroxypinacolone Retinoate and Physical Blend

Deionized water was added to a glass vessel containing powder. The vessel was capped and shaken vigorously for 2 minutes, then left to settle for 2 minutes. Water was yellow and powder was dispersed to form a cloudy solution for the complex HPR in gamma-cyclodextrin. Non-encapsulated HPR floated to the top of the vessel or stuck to the glass walls, creating an obvious opaque yellow layer for the physical blend (comparative example 16).

### Example 10: Method for Determining the pH of Complex CD/HPR/AGR

CD/HPR/AGR powder (2%) is added to a beaker and dispersed in deionized water (98%). The mixture is kept stirring with a stirring bar and a pH probe is placed in the center of the beaker. The pH is monitored by a pH meter until a stable reading is obtained. The pH of CD/HPR/AGR powder is between 2.0 and 6.0.

### Example 11: Method for Determining Bulk Density of Complex CD/HPR/AGR

CD/HPR/AGR powder is loaded into a bulk density meter and shaken into the graduated cylinder until it is full. The graduated cylinder is then leveled, and the weight of the sample is taken and divided by the volume of the cylinder. Care is taken not to tap down or pack any of the powder in the cylinder. The bulk density for CD/HPR/AGR is between 0.1 and 0.5 grams per milliliter.

### Example 12: Method for Determining Non-Volatile Content of Complex CD/HPR/AGR

An aluminum pan is weighed and 0.5 grams of the CD/HPR/AGR powder is added and weighed. The pan is placed in an oven at 105° C. for one hour. The sample pan is then removed from the oven and weighed again. The final weight is subtracted from the starting weight of the sample, and the non-volatile content is calculated as a percentage. The non-volatile content of CD/HPR/AGR is between 70 and 100%.

### Example 13: Method for Determining Water Activity (Aw) of Complex CD/HPR/AGR

The CD/HPR/AGR powder is placed in a plastic testing pan and placed in the water activity meter. The meter is then closed and the detector inside the instrument measures the vapor pressure of the sample and compares it to the vapor pressure of distilled water under the same exact conditions, i.e. airflow, temperature, and atmospheric pressure. The water activity of CD/HPR/AGR is between 0.1 and 0.5.

### Example 14: Cosmetic Formulation Containing Both HPR in Dimethyl Isosorbide (as Granactive Retinoid™) and Bakuchiyl Salicylate in Vitamin F Glyceryl Esters

This formula was created to showcase the stability of both HPR (I) and bakuchiyl salicylate (VII) combination. It is known that HPR is stable in formulas on its own, as is bakuchiyl salicylate. Little was known about their stability once formulated together. This formula serves as an example of the stability of both compounds when formulated together. Both active ingredients are added to the emulsion during the cooling process (phase C). This formula was tested by HPLC to ensure that HPR and bakuchiyl salicylate were still active, then a stability study was performed to determine their residual activity over a course of 4 weeks at 40° C. It was found that HPR remained above 75% active, while bakuchiyl salicylate remained 100% active after 4 weeks.

| Phase | Ingredients | INCI | % |
|---|---|---|---|
| A | DEIONIZED WATER | Water | 64.950 |
| | BUTYLENE GLYCOL | Butylene Glycol | 5.000 |
| | SORBIC ACID | Sorbic Acid | 0.200 |
| | SODIUM CITRATE | Sodium Citrate | 0.300 |
| | CITRIC ACID | Citric Acid | 0.100 |
| | GLYCERIN | Glycerin | 2.000 |
| | BIOWAVE NP | Water & Propanediol & Polyglutamic Acid & 1,2- | 3.000 |
| | | Hexanediol & Caprylyl Glycol | |
| | ARISTOFLEX VELVET | Polyacrylate Crosspolymer-11 | 0.500 |
| | AMPHISOL K | Potassium Cetyl Phosphate | 1.000 |
| | EUMULGIN SG | Sodium Stearoyl Glutamate | 0.750 |
| B | GRANSIL SBG-11 | Dimethicone & Polysilicone-11 & Butyrospermum | 10.000 |
| | | Parkii (Shea) Butter | |
| | RITA CA | Cetyl Alcohol | 0.750 |
| | BCR VEGAN LIPID | Squalane & Amaranthus Caudatus Seed Oil & | 0.200 |
| | COMPLEX | Hordeum Distichon (Barley) Extract & Triticum (Wheat) | |
| | | Germ Oil | |
| | INDOPOL H-100 | Polybutene | 0.500 |
| | JEECHEM GMS-450 | Glyceryl Stearate | 1.500 |
| | GRANPOWDER BBP- | Saccharomyces Ferment & Lauroyl Lysine | 1.250 |
| | 700 | | |
| | SQUALANE | Squalane | 1.000 |
| | SOFTISAN 649 | Bis-Diglyceryl Polyacyladipate-2 | 0.500 |
| | DIMETHICONE, 10 | Dimethicone | 3.000 |
| | CST | | |
| C | PROTACHEM CS-50 | Cetearyl Alcohol | 1.500 |
| | | Glyceryl Linoleate & Glyceryl Linolenate & Bakuchiyl | 1.000 |
| | | Salicylate (25% wt.) | |
| | GRANACTIVE | Dimethyl Isosorbide & Hydroxypinacolone Retinoate | 1.000 |
| | RETINOID ™ | | |
| | | Formula Total: | 100.000 |

### Procedure

1. Weigh Phase A in the main kettle equipped with homogenizer. Heat to 70-75 C.
2. Weigh Phase B in the side kettle. Heat to 70-75 C.
3. When at proper temperature, add Phase B to Phase A. Mix well.
4. Cool to 50 C with side sweep agitation and add Phase C. Mix well to uniform.
5. Continue cooling to room temperature.

### Example 15: Cosmetic Formulation Containing Both HPR Encapsulated in Gamma-CD with AGR (Example 3) and Bakuchiyl Salicylate in Vitamin F Glyceryl Esters

This formula was created to showcase the stability of both HPR encapsulated in gamma-cyclodextrin with AGR and bakuchiyl salicylate in vitamin F glyceryl ester combination. It is known that HPR is stable in formulas on its own, as is bakuchiyl salicylate. Little was known about their stability once formulated together, in the presence of AGR. This formula serves as an example of the stability of both compounds when formulated together. In this formula, complex HPR/CD/AGR powder is added to the water phase of the emulsion (phase A), which makes up 85.8% of the formula. Bakuchiyl salicylate is added to the oil phase of the emulsion (phase B), which makes up 13.7% of the formula. The percentage of water in this formula is 71.05%, suggesting that both the active ingredients are stable in formulations made with a high water-content. It was previously shown that high water content of >60% was challenging for HPR, even if encapsulated. There were no signs of formulation instability, such as precipitation or crystallization or separation in the formula. The formula passed a series of accelerated stability tests known to those in the art or formulation practice.

| Phase | Ingredients | INCI | % |
|---|---|---|---|
| A | DEIONIZED WATER | Water | 71.050 |
| | WHITE BIRCH NP | Water & Betula Alba Bark Extract & | 2.000 |
| | | Hydroxyacetophenone & | |
| | | Sodium Benzoate & 1,2-Hexanediol & Caprylyl | |
| | | Glycol | |
| | BIOWAVE NP | Water & Propanediol & Polyglutamic Acid & 1,2- | 3.000 |
| | | Hexanediol & Caprylyl Glycol | |
| | SYMDIOL 68 | 1,2-Hexanediol & Caprylyl Glycol | 1.000 |
| | SYMSAVE H | Hydroxyacetophenone | 0.250 |
| | ZEMEA | Propanediol | 5.000 |
| | GLYCERIN | Glycerin | 2.000 |
| | AMPHISOL K | Potassium Cetyl Phosphate | 0.500 |
| | Example 3 | Cyclodextrin & Hydroxypinacolone Retinoate & | 1.000 |
| | | Glucosylrutin | |
| B | CHEMJAC KX | Glucomannan & Xantham Gum | 0.500 |
| C | OLIVEM 1000 | Cetearyl Olivate & Sorbitan Olivate | 2.500 |
| | GRANSENSE TC-55X/C | Coco-Caprylate/Caprate & Caprylic/Capric | 8.000 |
| | | Triglyceride & C9-12 | |
| | | Alkane & Dilinoleic Acid/Butanediol Copolymer & | |
| | | Castor Oil/IPDI Copolymer | |
| | BCR VEGAN LIPID | Squalane & Amaranthus Caudatus Seed Oil & | 0.200 |
| | COMPLEX | Hordeum Distichon (Barley) Extract & Triticum (Wheat) | |
| | | Germ Oil | |
| | RITAPHYL ICS | Isocetyl Stearate | 1.000 |
| | GRANPOWDER BBP- | Saccharomyces Ferment & Lauroyl Lysine | 1.000 |
| | 700 | Glyceryl Linoleate & Glyceryl Linolenate & Bakuchiyl | 1.000 |
| | | Salicylate (25% wt) | |
| | | Formula Total: | 100.000 |

### Procedure

1. Weigh Phase A in the main kettle equipped with homogenizer. Mix well.
2. Sprinkle Phase B into Phase A. Mix well and heat to 70-75 C.
3. In a side kettle, mix and heat Phase C to 75-80 C.
4. Add Phase C to Phase AB. Mix well.
5. Cool to room temperature with side sweep agitation.

### Comparative Example 16: Physical Blend of Hydroxypinacolone Retinoate and Gamma-Cyclodextrin

Hydroxypinacolone retinoate (I) (10%) and gamma-cyclodextrin (90%) was added to a standing KitchenAid™ mixer (Model No. KSM150PSMC) equipped with a flat beater mixer. This obtained a free-flowing powder after several minutes of mixing. The product had poor color stability as indicated by a rapid loss of color from bright yellow to white within 1 week. This was an indication of the degradation of the HPR. This blend was added to water and shaken (example 9), the HPR (I) crystals floated on top of the water and were not dispersible, therefore indicating encapsulation was unsuccessful. This example shows that a physical blend of the ingredients is insufficient to provide a useful composition. It also demonstrates the need for proper processing and for the encapsulation of the HPR to be water-dispersible.

### Comparative Example 17: Opposite Order of Addition Compared to Example 2

In standing KitchenAid™ mixer (Model No. KSM150PSMC) equipped with a flat beater, Hydroxypinacolone retinoate (HPR) (10%) was dispersed in isopropanol and heated to less than 80° C. until dissolved and the solution is clear yellow (Phase A). Gamma-Cyclodextrin (55%) was added to a beaker with a mixer and water (45%) was added and mixed (Phase B). Phase B (77%) was added to Phase A (23%) and kneaded for 30 minutes to a slurry. During this process, a dilatant phase is not reached. The slurry showed visible HPR (1) crystals sitting atop the mixture and sticking to the walls of the mixer bowl. The slurry was scooped out of the mixer bowl and spread evenly onto oven safe trays. The trays were placed in an oven at 65° C. After about 18 hours, the powder cakes were tested for dryness by moisture balance and transferred to a mill to obtain a free-flowing powder. The resulting powder was brighter yellow in color and smelled strongly of HPR. Upon HPLC testing (example 8) and qualitative encapsulation testing (example 9), it was found that HPR was not efficiently encapsulated in gamma-cyclodextrin. This mixture blend was added to water and shaken, the HPR (I) crystals floated on top of water and were not dispersible, therefore indicating encapsulation was unsuccessful. This example therefore shows that order of addition is critical to achieving the product and that incorrect processing order fails to yield the product.

### Comparative Example 18: Complexation of Retinol in Gamma-Cyclodextrin (CD/Retinol)

In a beaker, retinol (CAS 68-26-8) (10%) was dispersed in isopropanol (90%) and heated to less than 80° C. until dissolved and the solution is clear yellow (Phase A). Gamma-Cyclodextrin (55%) was added to a standing KitchenAid™ mixer (Model No. KSM150PSMC) equipped with a flat beater and water (45%) was added and mixed (Phase B). Phase B can behave as a non-Newtonian fluid at concentrations of 40-70%. Phase A (17%) was added to Phase B (83%) and kneaded for 30 minutes to a dough-like consistency or slurry. During the mixing process of Phase A into Phase B, a dilatant phase (shear-thickening) is reached but is then surpassed as Phase A is continually added to Phase B. The slurry was scooped out of the mixer bowl and spread evenly onto oven safe trays. The trays were placed in an oven at 65° C. After about 16 hours, the powder cakes were tested for dryness by moisture balance and transferred to a mill to obtain a free-flowing powder. The powder is expected to have a loss of drying of less than 15%.

### Comparative Example 19: Complexation of Retinaldehyde in Gamma-Cyclodextrin (CD/Retinaldehyde)

In a beaker, retinaldehyde (CAS 116-31-4) (10%) was dispersed in isopropanol (90%) and heated to less than 80° C. until dissolved and the solution is clear yellow (Phase A). Gamma-Cyclodextrin (55%) was added to a standing KitchenAid™ mixer (Model No. KSM150PSMC) equipped with a flat beater and water (45%) was added and mixed (Phase B). Phase B can behave as a non-Newtonian fluid at concentrations of 40-70%. Phase A (16.9%) was added to Phase B (83.1%) and kneaded for 30 minutes to a dough-like consistency or slurry. During the mixing process of Phase A into Phase B, a dilatant phase (shear-thickening) is reached but is then surpassed as Phase A is continually added to Phase B. The slurry was scooped out of the mixer bowl and spread evenly onto oven safe trays. The trays were placed in an oven at 65° C. After about 16 hours, the powder cakes were tested for dryness by moisture balance and transferred to a mill to obtain a free-flowing powder. The powder is expected to have a loss of drying of less than 15%.

### Comparative Example 20: Complexation of Retinyl Palmitate in Gamma-Cyclodextrin (CD/Retinyl Palmitate)

In a beaker, retinyl palmitate (CAS 79-81-2) (10%) was dispersed in isopropanol (90%) and heated to less than 80° C. until dissolved and the solution is clear yellow (Phase A). Gamma-cyclodextrin (55%) was added to a standing KitchenAid™ mixer (Model No. KSM150PSMC) equipped with a flat beater and water (45%) was added and mixed (Phase B). Phase B can behave as a non-Newtonian fluid at concentrations of 40-70%. Phase A (27.9%) was added to Phase B (72.1%) and kneaded for 30 minutes to a dough-like consistency or slurry. During the mixing process of Phase A into Phase B, a dilatant phase (shear-thickening) is reached but is then surpassed as Phase A is continually added to Phase B. The slurry was scooped out of the mixer bowl and spread evenly onto oven safe trays. The trays were placed in an oven at 65° C. After about 16 hours, the powder cakes were tested for dryness by moisture balance and transferred to a mill to obtain a free-flowing powder. The powder is expected to have a loss of drying of less than 15%.

### Example 21: Dispersibility in Water of HPR in Excipients

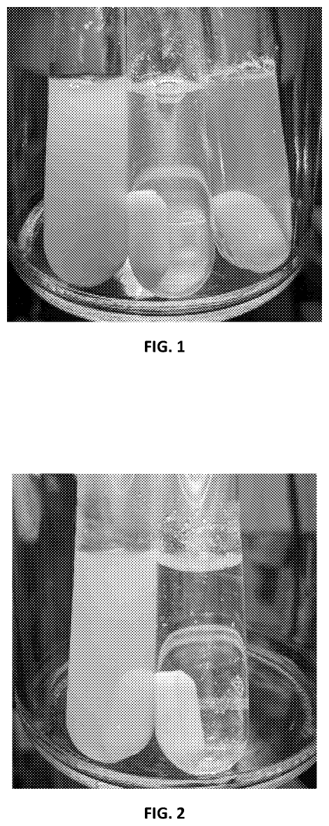

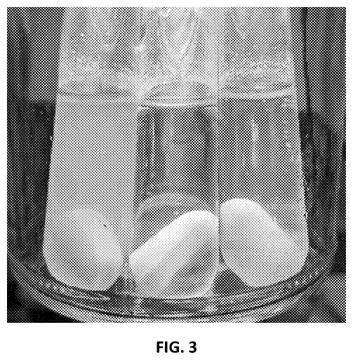

A spatula tip amount (0.05 g) of HPR in DMI, Example 2 and Comparative Example 16 were added to separate test tubes with water (5 mL) and stirred to observe the uniformity of the mixture. As shown in FIGS. 1-3, it was clearly observed with bare eyes (no microscopic analysis) that HPR in DMI and Comparative Example 16 had precipitation of HPR crystals at 25° C. and at 85° C. after 15 minutes. It was clearly observed that Example 2 (complex of HPR/g-CD) was stable and easily dispersed in water with no visible signs of crystals at 25° C. and 85° C. for 15 minutes. It was observed that only after 2.5 hours at 85° C. that crystals of HPR were slowly releasing from the g-CD complex. This example shows a comparison between GAR (prior art), physical blend (comparative example showing processing importance), and most importantly the water dispersibility of CD/HPR/AGR. No crystallization shows ease of use and proves encapsulation benefits.

TABLE 3
Dispersibility in water of HPR in excipients

| | | 85° C. | 85° C. |
|---|---|---|---|
| Dispersibility in water | 25° C. | (15 min) | (2.5 hrs) |
| HPR in DMI | No | No | N/A |
| Example 2 | Yes | Yes | Yes, |
| | | | with some |
| | | | release of HPR |
| Comparative Example 16 | No | No | N/A |

While the invention has been described above with examples to specific embodiments thereof, it is impossible to cover all aspects of the invention. Many changes, modifications and variations with the compositions of the invention will thereof be obvious to those skilled in the art. All of which are within the spirit and scope of the invention without desertion of the inventive concept disclosed herein.

We claim:

1. A retinoid-containing composition with extended storage stability comprising: hydroxypinacolone retinoate (HPR) complexed within a cyclodextrin.

2. The retinoid-containing composition of claim 1, wherein the cyclodextrin is gamma (v) cyclodextrin.

3. The retinoid-containing composition of claim 1, wherein the hydroxypinacolone retinoate complexed with the cyclodextrin is in an amount from about 0.01% to about 25% by weight of the composition.

4. The retinoid-containing composition of claim 3, wherein the amount of hydroxypinacolone retinoate complexed with the cyclodextrin is from about 1% to about 10% by weight of the composition.

5. The retinoid-containing composition of claim 1, wherein the cyclodextrin is in an amount from about 80% to about 98% by weight of the composition.

6. The retinoid-containing composition of claim 5, wherein the amount of cyclodextrin is from about 85 to about 95% by weight of the composition.

7. The retinoid-containing composition of claim 1, further comprising an antioxidant.

8. The retinoid-containing composition of claim 7, wherein the antioxidant is a flavonoid.

9. The retinoid-containing composition of claim 8, wherein the flavonoid is selected from the group consisting of quercetin, rutin flavonoids, glutathione, tocopherols (Vitamin E), ascorbic acid (Vitamin C), butylated hydroxyanisole (BHA), butylated hydroxytoluene (BHT), uric acid, and ubiquinol.

10. The retinoid-containing composition of claim 9, wherein the rutin flavonoid is alpha-glucosyl rutin.

11. The retinoid-containing composition of claim 1, wherein said composition is a water dispersible composition without surfactants.

12. The retinoid-containing composition of claim 7, wherein the antioxidant is in an amount from about 1% to about 10% by weight of the composition.

13. The retinoid-containing composition of claim 12, wherein the amount of antioxidant is from about 3% to about 8% by weight of the composition.

14. The retinoid-containing composition of claim 9, wherein the cyclodextrin is gamma (v) cyclodextrin, the antioxidant is the flavonoid alpha-glucosyl rutin (AGR).

15. The retinoid-containing composition of claim 14, wherein the HPR is present in an amount of 0.1-25% by weight of the composition, the γcyclodextrin is in an amount of 75-98.9% by weight of the composition, and the AGR is in an amount of 1-10% by weight of the composition.

16. The retinoid-containing composition of claim 1, wherein the HPR maintains RARE activity of at least 97% without the use of a container with inert atmosphere storage after 3 months at 45 degrees C.

17. A topical cosmetic or pharmaceutical composition comprising the retinoid-containing composition of claim 1.

18. The topical cosmetic or pharmaceutical composition of claim 17, wherein the retinoid-containing composition is present in an amount from about 0.001% to about 1% by weight of the topical cosmetic or pharmaceutical composition.

19. The topical cosmetic or pharmaceutical composition of claim 18, wherein the amount of the retinoid-containing composition is from about 0.01% to about 0.5% by weight of the topical cosmetic or pharmaceutical composition.

20. The topical cosmetic or pharmaceutical composition of claim 17, further comprising a bioactive enhancer.

21. The topical cosmetic or pharmaceutical composition of claim 20, wherein the bioactive enhancer is a bakuchiol ester.

22. The topical cosmetic or pharmaceutical composition of claim 21, wherein the bakuchiol ester is bakuchiol salicylate.

23. The topical cosmetic or pharmaceutical composition of claim 20, wherein the bioactive enhancer is present in an amount from about 0.005% to about 0.3% by weight of the composition.
